(19) United States
(12) Patent Application Publication
(10) Pub. No.: US 2023/0364282 A1
(43) Pub. Date: Nov. 16, 2023

(54) ELECTRICALLY ENERGIZED DOOR LATCH BOX FOR TRANSFERRING ELECTRIC POWER THROUGH A DOOR LOCKSET

(71) Applicant: Tom Chi, Wichita, KS (US)

(72) Inventor: Tom Chi, Wichita, KS (US)

(21) Appl. No.: 17/913,053

(22) PCT Filed: Mar. 25, 2021
(86) PCT No.: PCT/US2021/024100

§ 371 Date: Sep. 20, 2022

Related U.S. Application Data
(60) Provisional application No. 63/000,602, filed on Mar. 27, 2020.

(51) Int. Cl. A61L 2/10; E05B 1/00; A61L 2/26

(52) U.S. Cl. CPC A61L 2/10; E05B 1/0069; A61L 2/26; A61L 2202/11

(57) ABSTRACT

An electrically energized door latch box for transferring electric power through a door lock-set to a door containing an electric device includes a door for entering and exiting a location, a device for transmitting electrical power to the door, and at least one door-powered electrical device associated with the door. A switch for switching on and off the electrically energized door and a movable lock barrel for activating the switch are also included.

### BACKGROUND OF THE INVENTION

### Technical Field of the Invention

The present invention relates, generally, to supplying electric power to doors in order for the doors to electrically connect with, and energize, door associated devices that require electrical energy, such as ultraviolet C light enabled doorknobs, door handles, door levers for sterilization of the touching surfaces, fans, security systems, power lights, emergency exit lights, emergency signs, rechargeable batteries, video displays and other devices, with the primary purpose of the invention to easily transfer power to the devices.

### Description of the Prior Art

It is well established that germs from hands are easily communicated to a person's face especially when the hands touch the face several times a minute as, for example, when the eyes are rubbed, the nose is touched and other times when the hands consciously and unconsciously touch the face.

Since the exposed face is more than any other part of the human body more susceptible to transmitting germs to the body's systems it imperative to maintain as best as possible a germ-free face and hands-free of pathogens, which can enter the eyes, ears, mouth, nose etc.

The prior art known to the inventor is represented by the following: Littles, U.S. Pat. No. 4,777,570, issued Oct. 11, 1988; Geringer et al., U.S. Patent Application Publication No. 2016/0273243, published Sep. 22, 2016; Martin et al., U.S. Patent Application Publication No. 2018/0080253, published Mar. 22, 2018; Martin et al., U.S. Patent Application Publication No. 2018/0171669, published Jun. 21, 2018; and, Gerhardt et al., U.S. Patent Application Publication No. 2019/0342443.

### SUMMARY OF THE INVENTION

A primary object of the present invention is to easily and effectively supply electric power to a door in order for the door to electrically connect with and energize ultraviolet light C enabled door knobs and door handles for surface sterilization or other purposes that require a transition of electric power from a door frame to a door for recharge rechargeable batteries, power lights, smoke alarms, emergency exit lights, thru-door fans, video displays and similar devices with the primary purpose of the present invention to provider a source of electrical energy to door devices.

A further object of the present invention is to provide a wiring system internal to the door jamb so that the electric wires are “built-in” to the door jamb.

Another object of the present invention is to internally locate the electric wires so that the energy source is not exposed.

Still another object of the invention is to locate an “on-off” switch within the door latch box and to be activated when the lock barrel engages with the electrical switch.

Another object of the present invention is to provide way for converting standard 120-volt and/or standard 240-volt power to low DC power for safety considerations.

A still further object of the invention is to provide means so that the system may be automatically deactivated to shutoff when there is an absence of contact with a switch contained in the door latch box and automatically activate when the door closes and the lock barrel engages with the switch.

An additional object of the present invention is to utilize the power supplied to the door for any purpose, such as, for observational cameras, for night and other lighting and may include energizing ultraviolet lights for sterilization of doorknobs, pull handles, door levers, door push plates etc., for the purpose of maintaining sterility and decontaminating contaminated surfaces from germs.

Another object of the present invention is to provide for use on a keyed or non-keyed deadbolt with any type of handle or any latching mechanism including but not limited to magnetic latches etc.

A still further object of the present invention is to provide a lockset that may be used with a push-button lockset, phone-controlled lockset or other type of locking or latching mechanism.

Yet, a further object of the present invention permits the conveyed power for use with automatic locks and other devices.

A still further object is to provide battery power on either side of the door jamb or on the door itself.

The foregoing and related objects of the present invention are accomplished by an electrically energized door latch box for transferring electric power through a door lock-set to a door containing an electric device, which includes a door for entering and exiting a location, means for transmitting electrical power to the door, and at least one door-powered electrical device associated with the door. A switch for switching on and off the electrically energized door and a movable lock barrel for activating the switch are also included.

Preferably, one or more door-powered electrical devices associated with the door includes an ultraviolet C light-enabled doorknob, an ultraviolet C light enabled-door handle, an ultraviolet C light-enabled door lever, an ultraviolet C light-enabled door push plates for sterilization of the touching surfaces, a fan, a security system, a powered light, emergency exit lights, emergency signs, a rechargeable battery, a video display, in addition to one or more other advantageous devices that would require electrical energy.

Preferably, the switch transfers electric power to the door through lock barrel engaged with switch for energizing the door powered electrical devices.

Preferably, the ultraviolet C light-enabled doorknobs, ultraviolet C light-enabled door handles, ultraviolet C light-enabled door levers and ultraviolet C light enabled door push plates for sterilization are enabled and include light emitting diodes, fiber optics, glass fiber and mercury bulbs.

Ultraviolet light enabled doorknobs and door handles maintain a germ-free surface by disinfecting and sterilizing doorknobs, door handle levers, door bar handles and other devices that are touched by a user's hands. By exposing the doorknob, door lever, door bar handle, faucet lever/handle, elevator buttons and other devices to ultraviolet sterilizing light to kill or inactivate microorganisms by destroying nucleic acids, disrupting their DNA/RNA and leaving them unable to function or reproduce.

Other objects and features of the present invention will become apparent when considered in combination with the accompanying drawing figures, which illustrate certain preferred embodiment of the present invention. It should, however, be noted that the accompanying drawing figures are intended to illustrate only select preferred embodiments of the claimed invention and are not intended as a means for defining the limits and scope of the invention.

### BRIEF DESCRIPTION OF THE DRAWING FIGURES

In the drawing figures, wherein similar features are denoted with similar reference numerals throughout the several views:

FIGS. 1 and 1A provide perspective views with various alternative preferred embodiments for practicing the present invention when the system of the present invention is not activated and the electric power is not being transmitted to the door; and,

FIGS. 2 and 2A provide perspective views with various alternative preferred embodiments for practicing the present invention when the system of the present invention is activated and the electric power is being transmitted to the door.

### DESCRIPTION OF THE REFERENCE NUMERALS

100a present invention of non-energized door latch box
100b present invention of energized door latch box
102 stationary door frame
104 door for entering and exiting a premise
106a ultraviolet light emitting doorknob
106b ultraviolet light emitting contoured door lever
106c ultraviolet light emitting rectangular door lever
106d ultraviolet light emitting door bar
106e ultraviolet light emitting door push plate
108 non-engaged lock barrel
108a lock barrel engaged with switch that provides electric power to ultraviolet

C light enabled doorknobs and other door devices
110 door latch box containing electrical switch
112 electrical switch and when activated transmits electric power through lock barrel to door
114 electrical energy source, generally, 110-volt AC power source
116 AC-to-DC converter
118 electrical port
120 ultraviolet C sanitizing light

### DETAILED DESCRIPTION OF THE DRAWING FIGURES AND PREFERRED EMBODIMENTS

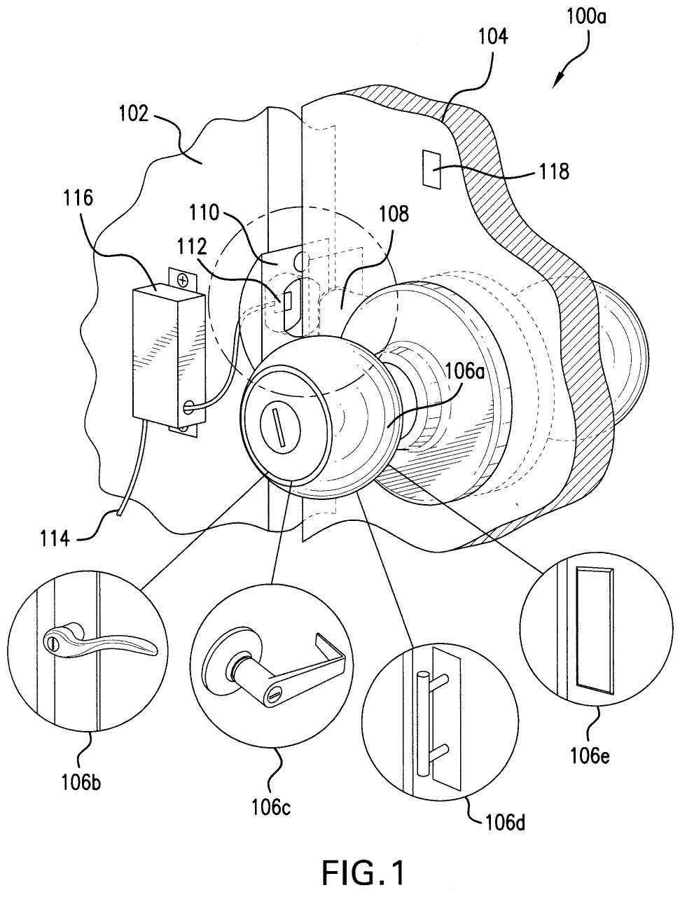

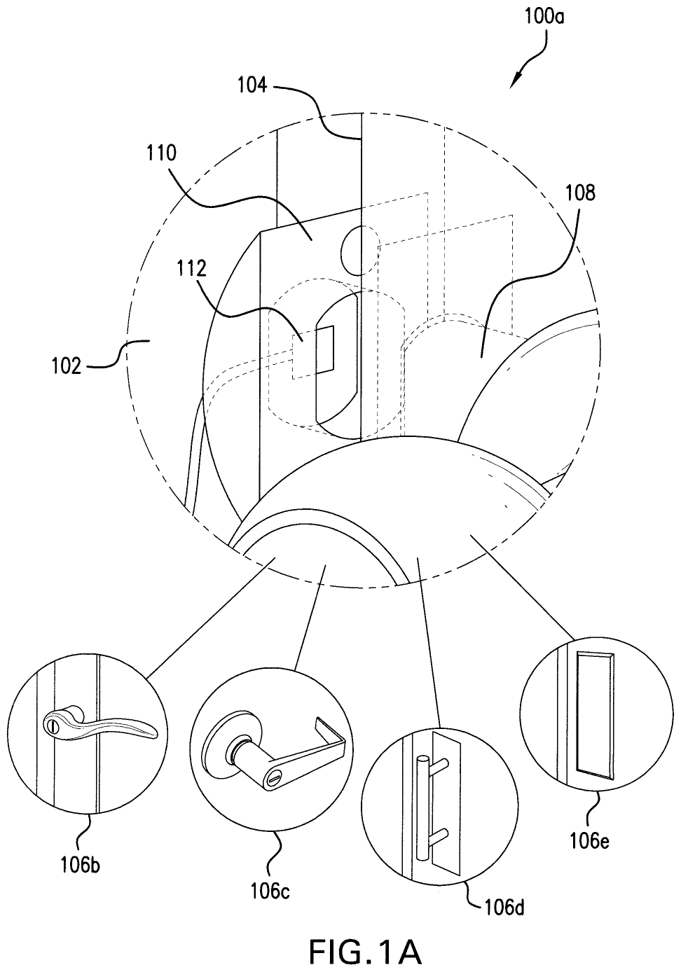

FIG. 1 illustrates a non-energized door latch box 100a and details the method of transmitting of power 114 (conventionally 120 volts AC but not limited thereto) by means of an AC-to-DC converter 116 from a stationary door frame 102 to a door 104. Door latch box 110 contains an electrical switch 112 and, as shown, is not activating electrical switch 112 and accordingly electricity does not flow to door 104.

FIG. 1A illustrates an additional view of a non-energized door latch box 100a and details the method of transmitting of power 114 (conventionally 120 volts AC but not limited thereto) by means of AC-to-DC converter 116 from stationary door frame 102 to door 104. Door latch box 110 contains electrical switch 112 and as shown is not activating electrical switch 112 and according electricity does not flow to door 104.

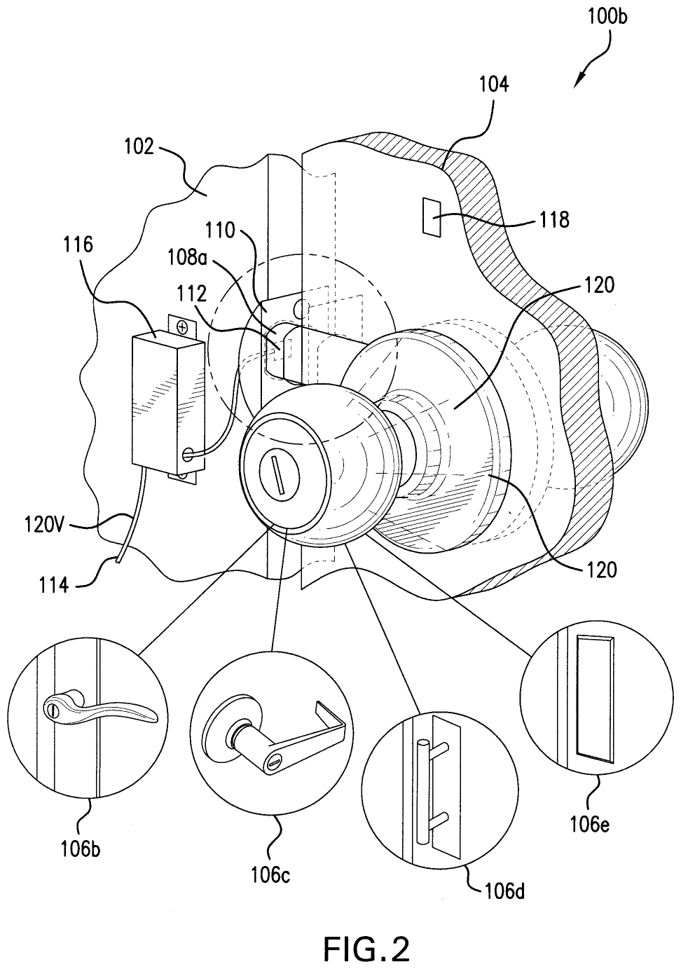

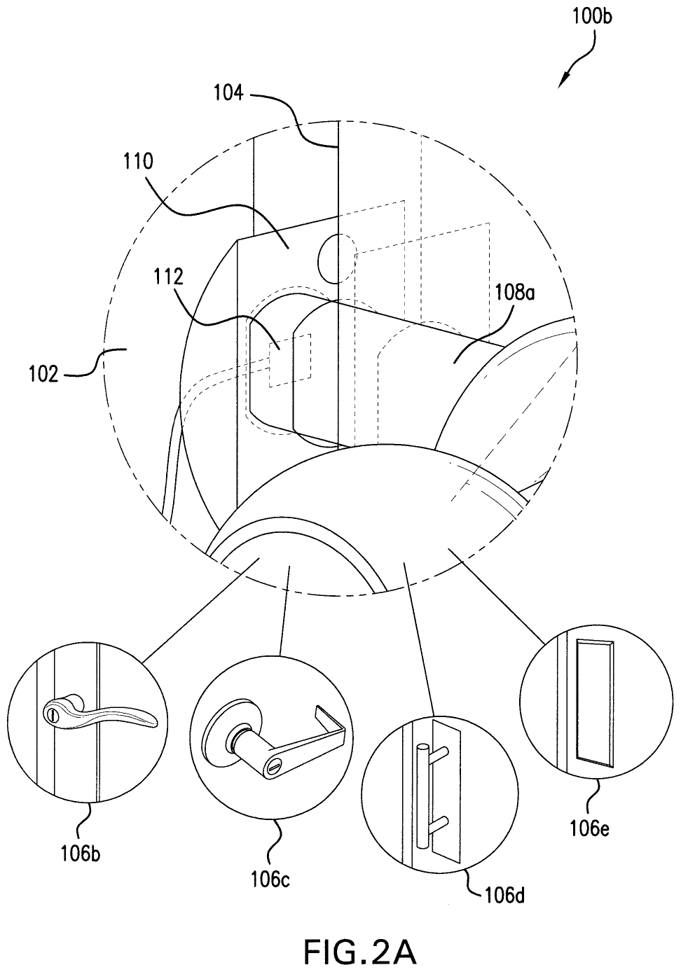

FIG. 2 illustrates an energized door latch box 100b and the method of transmitting of power 114 (conventionally 120 volts AC but not limited thereto) by means of AC-to-DC converter 116 from stationary door frame 102 to door 104. When the door is closed, as shown, engaged lock barrel 108a engages electrical switch 112 and accordingly electric current flows through lock barrel 108a from electrical switch 112 into door 104 for electrical energy access. Door latch box 110 contains electrical switch 112 and when electrical switch 112 is activated by engaging with lock barrel 108a electricity is able to flow into engaged lock barrel 108a and then to door 104, energizes an ultraviolet C germicidal light source incorporated into an ultraviolet C light emitting doorknob 106a, energizes ultraviolet C light emitting contoured door lever 106b, energizes ultraviolet C light emitting rectangular door lever 106c, energizes ultraviolet C light emitting door bar 106d, an energizes ultraviolet C light emitting door push plate 106e, energizes electrical port 118, and other door-associated devices such as, fans, security systems, power lights, emergency exit lights, emergency signs, rechargeable batteries, video displays and other devices that require electrical energy. The ultraviolet C light enabled doorknobs, ultra-violet C light enabled door handles, ultraviolet C light enabled door levers and ultraviolet C light enabled door push plates for sterilization are enabled and includes light emitting diodes, fiber optics, glass fiber and mercury bulbs. Electrical port 120 is incorporated into door 104 for access to electrical energy.

FIG. 2A provides a further view of the energized door latch box 100b and the method of transmitting of power 114 (typically 120 volts AC but not limited thereto) by means of an AC-to-DC converter 116 from stationary door frame 102 to door 104. When the door is closed, as shown, the engaged lock barrel 108a engages an electrical switch 112 and accordingly electric current flows through lock barrel 108a from the electrical switch 112 into the door 104 for electrical energy access.

Door latch box 110 contains electrical switch 112 and when electrical switch 112 is activated by engaging with lock barrel 108a electricity to flow into engaged lock barrel 108a and then to door 104 and energizes ultraviolet C germicidal light source incorporated into ultraviolet C light emitting doorknob 106a energizes ultraviolet C light emitting contoured door lever 106b, energizes ultraviolet C light emitting rectangular door lever 106c, energizes ultraviolet C light emitting door bar 106d, energizes ultraviolet C light emitting door push plate 106e, energizes electrical port 118 and other door associated devices such as, fans, security systems, power lights, emergency exit lights, emergency signs, rechargeable batteries, video displays and other devices that require electrical energy. The ultraviolet C light enabled doorknobs, ultraviolet C light enabled door handles, ultraviolet C light enabled door levers and ultraviolet C light enabled door push plates for sterilization are enabled and includes light emitting diodes, fiber optics, glass fiber and mercury bulbs. Electrical port 120 is incorporated into door 104 for access to electrical energy.

While only several embodiments of the present invention have been shown and described, it will be obvious to those skilled in the art that many modifications may be made to the present invention without departing from the spirit and scope thereof.

1. An electrically energized door latch box for transferring electric power through a door lockset to a door containing electric devices, comprising:
a door for entering and exiting a premise;
a door-powered electrical device associated with the door, comprising a light-emitting diode, fiber optics, glass fibers and a mercury bulb for sterilizing at least one of the door and the door-powered electrical device associated with the door;
an activation switch to electrically energize the door;
a movable lock barrel for activating the activation switch; and,
means for transmitting electrical power to the door.

2. The electrically energized door latch box for transferring electric power through a door lockset to a door containing electric devices according to claim 1, wherein the door powered electrical device associated with the door is at least one of an ultraviolet C light enabled doorknob, an ultraviolet C light enabled door handle, an ultraviolet C light enabled door lever and an ultraviolet C light enabled door push plate for sterilizing a surface that requires electric power.

3. (canceled)

4. The electrically energized door latch box for transferring electric power through a door lockset to a door containing electric devices according to claim 1, wherein the activation switch causes a transfer of electric power to the door through the movable lock barrel engaged with the activation switch for energizing the door powered electrical device.

5. The electrically energized door latch box for transferring electric power through a door lockset to a door containing electric devices according to claim 1, further comprising an electrical port contained in the door for electrical energy access.
